(19) United States
(12) Patent Application Publication
(10) Pub. No.: US 2005/0228406 A1
(43) Pub. Date: Oct. 13, 2005

(54) Formation of knots

(76) Inventor: Ganendra Coomer Bose, Dalkeith, Western Australia (AU)

Correspondence Address: BACHMAN & LAPOINTE, P.C., 900 CHAPEL STREET, SUITE 1201, NEW HAVEN, CT, 06510, US

(21) Appl. No.: 10/519,883

(22) PCT Filed: Jul. 3, 2003
(86) PCT No.: PCT/GB03/02847

§ 371 Date: Dec. 30, 2004

(30) Foreign Application Priority Data
Jul. 3, 2002 (AU) PS 3320
Oct. 21, 2002 (AU) 2002952166

(51) Int. Cl. A61B 17/04; A61B 17/12

(52) U.S. Cl. 606/144

(57) ABSTRACT

The present invention is concerned with the formation of knots in filamentary material. The invention has particular application to the formation of knots in suturing and in ligating tubular structures, such as blood vessels. Devices are provided which carry at least one loop (22, 220, 230, 101, 116, 122) of filamentary material, such as suture material. The loop or loops can be used in the formation of sutures.

This invention relates to devices for use in the formation of knots in filamentary material and to methods of making knots. The invention has particular application to the formation of knots in suturing and in ligating tubular structures. The invention is not, however, limited to such uses and can find applications other than in conjunction with the human or animal body.

Laparoscopic suturing, ligating and knot tying is widely recognised as technically difficult. It takes a considerable amount of practice to master the technique and unless a surgeon has had enough practice in performing laparoscopic suturing or ligation regularly, the surgeon cannot do it easily or in a timely fashion during the operation.

In particular, it is difficult to deliver a curved needle for laparoscopic intracorporeal suturing in a desirable position such that it can be held and manipulated. Further, the tying of intracorporeal knots, either during suturing or ligating, can be extremely difficult and time consuming.

There are several special instruments and techniques, both automatic and semi-automatic, which have been developed for laparoscopic suturing over the years. Although many of them are technically superb, their use is cost prohibitive and limited mainly to the private health system.

The present invention attempts to overcome at least in part some of the aforementioned disadvantages.

According to a first aspect of the present invention there is provided a laparoscopic needle delivery device, comprising a generally elongate member, the member having a needle carrying portion for releasably carrying a needle and a loop carrying portion for releasably carrying at least one loop of filamentary material, said loop carrying portion being constructed and arranged to enable said at least one loop to be released from the elongate member whilst still formed as a loop.

According to a second aspect of the present invention there is provided a combination of:
the device of the above first aspect of the present invention;
said needle; and
a length of said filamentary material, wherein the needle is attached to the distal end of the length of filamentary material, the length of filamentary material has said at least one loop formed therein at a position spaced from said distal end, said at least one loop is engaged with said loop carrying portion so as to be carried thereby and said needle is engaged with said needle carrying portion so as to be carried thereby.

The combination of the above second aspect of the present invention may be used to form a suture, the method comprising releasing the needle from the needle delivery device, passing the needle through the elements to be sutured, passing the needle through the centre of said at least one loop and tensioning said at least one loop to cause said loop to contract to form a knot in the filamentary material.

According to a third aspect of the present invention there is provided a laparoscopic device for use in ligating a tubular structure, the device comprising an elongate member having a generally hook-shaped distal portion to enable the tubular structure that is to be ligated to be received in said portion, said elongate member further comprising a loop carrying portion for releasably carrying at least one loop of filamentary material, said loop carrying portion being constructed and arranged to enable said at least one loop of material to be released from the elongate member whilst still formed as a loop.

According to a fourth aspect of the present invention there is provided a combination of:
the device of the above third aspect of the present invention; and
a length of said filamentary material, wherein said length of filamentary material has said at least one loop formed therein and said at least one loop is engaged with said loop carrying portion so as to be carried thereby.

The combination of the above fourth aspect of the present invention used to ligate a tubular structure, the method comprising engaging with said generally hook-shaped distal portion the tubular structure to be ligated, passing the distal end of the filamentary material around the tubular structure and back through the centre of said at least one loop and tensioning said at least one loop to ligate the tubular structure and to form a knot.

According to fifth aspect of the present invention there is provided a laparoscopic needle delivery device, comprising a generally elongate member, the member being provided with a loop carrying portion for releasably carrying at least one loop of filamentary material and a needle carrying portion for releasably carrying a needle in both a longitudinal orientation, in which the needle is generally aligned with the longitudinal axis of the elongate member, and a transverse orientation, in which the needle is generally perpendicular to said longitudinal axis.

According to a sixth aspect of the present invention there is provided a combination of:
the device of the above fifth aspect of the present invention;
said needle; and
a length of said filamentary material, wherein the needle is attached to the distal end of the length of filamentary material, the length of filamentary material has said at least one loop formed therein at a position spaced from said distal end, said at least one loop is engaged with said loop carrying portion so as to be carried thereby and said needle is engaged with said needle carrying portion so as to be carried thereby.

The combination of the above sixth aspect of the present invention may be used to form a suture, the method comprising:
removing said needle from an initial needle position in said needle carrying portion;
passing the needle through the structure to be sutured, the centre of said at least one loop, and back into the needle carrying portion to assume a subsequent needle position;
releasing said at least one loop from said loop carrying portion to pass down the needle; and
tensioning said at least one loop to form and knot the suture.

According to a seventh aspect of the present invention there is provided a method of ligating a tubular structure, the method comprising:
providing a length of filamentary material having at least one loop formed therein spaced proximally from the filamentary material's distal end;
positioning said at least one loop adjacent the tubular structure to be ligated;
passing the distal end of the filamentary material around the tubular structure and then through the centre of said at least one loop; and
tensioning said at least one loop to ligate the tubular structure and to form a knot in the filamentary material.

According to an eight aspect of the present invention there is provided a method of forming an additional knot to secure a prior knot formed in a continuous length of filamentary material, a proximal portion of the filamentary material extending between a generally elongate first instrument and the prior knot and a distal portion of said filamentary material extending between the prior knot and the distal end of the filamentary material, the method comprising:
a) manipulating the first instrument and a second generally elongate instrument to form a loop in the proximal portion of the filamentary material, with said second generally elongate instrument extending through said loop;
b) gripping the distal portion of the filamentary material using said second instrument;
c) withdrawing the second instrument through said loop to draw the distal portion of the filamentary material through said loop; and
d) tensioning said loop to form said additional knot on top of said prior knot.

In the method of the above eighth aspect of the present invention the step (a) may involve the following sub-steps:
e) manipulating the first instrument to form said loop in said proximal portion of the filamentary material; and
f) extending the second instrument through said loop.

The present invention will now be described, by way of example only, with reference to the accompanying drawings, in which:

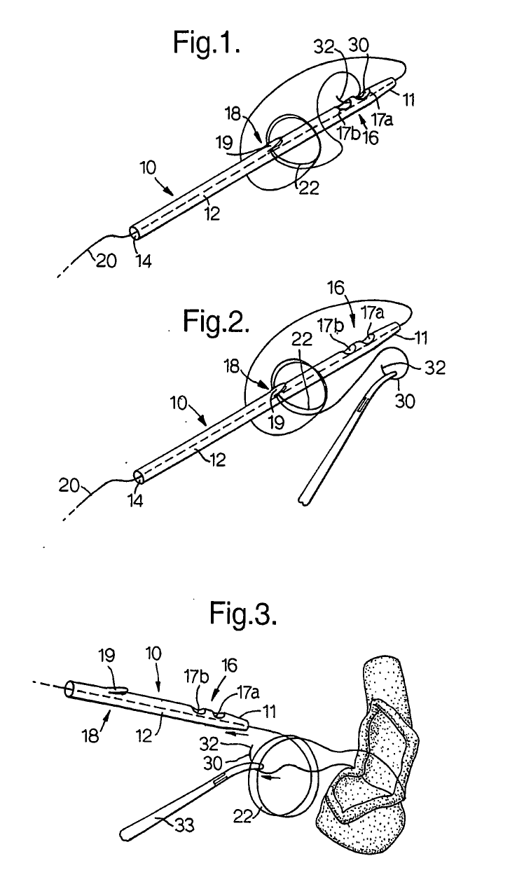

FIG. 1 is a diagrammatic perspective view of a laparoscopic needle delivery device in accordance with one aspect of the present invention;

FIG. 2 is a diagrammatic perspective view of the device shown in FIG. 1 wherein a needle, previously received by the device, is grasped and removed by a grasping device, and or a needle holder;

FIG. 3 is a diagrammatic perspective view of the device shown in FIGS. 1 and 2 wherein the needle has been passed through cut edges of a viscera by the grasping device and is shown being pulled through a pair of loops of suture, previously received by the device, by the grasping device in order to form a knot;

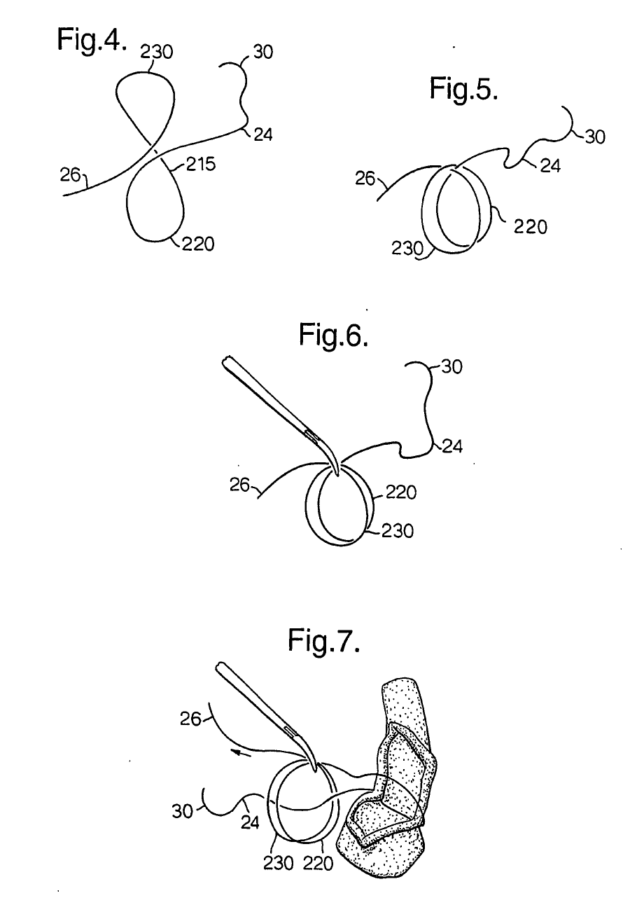

FIGS. 4 to 17 are schematic views of successive steps in forming and tying several intracorporeal knots with the aid of a similar kind of device as the laparoscopic needle delivery device of the present invention without a needle receiving means;

FIG. 18 is a perspective view of a loose knot, having an additional knot formed on top for extra security;

FIG. 19 is a diagrammatic view of a laparoscopic suturing device for ligating blood vessels or tubular structures in accordance with a second aspect of the present invention.

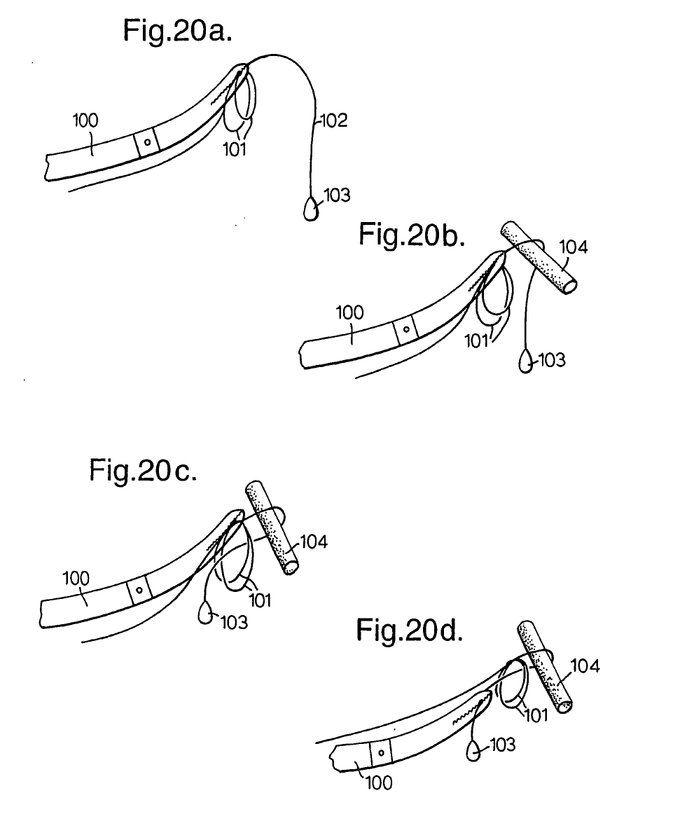

FIGS. 20a-20c are schematic views of successive steps in forming and tying an intracorporeal knot to ligate a tubular structure;

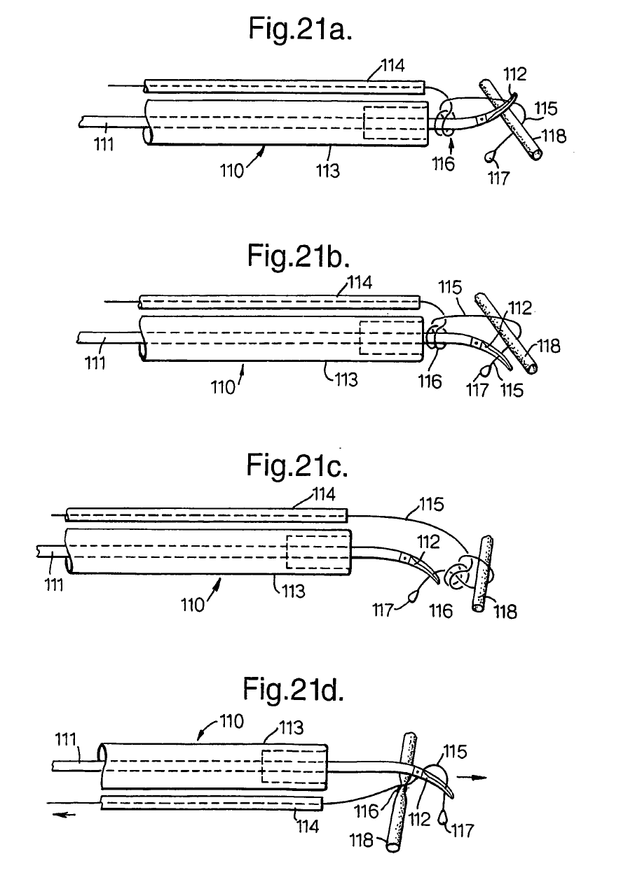

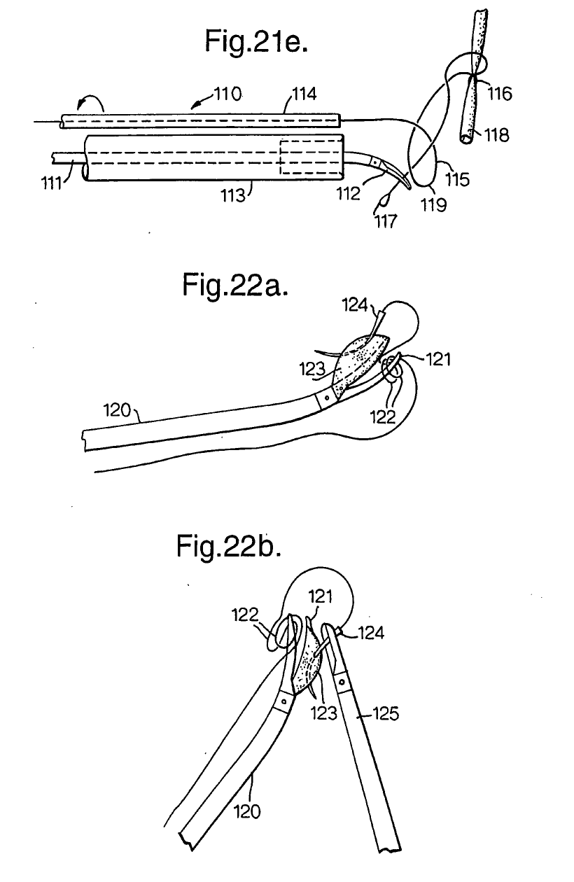

FIGS. 21a-21d are schematic views of successive steps in forming and tying an intracorporeal knot to ligate a tubular structure; and

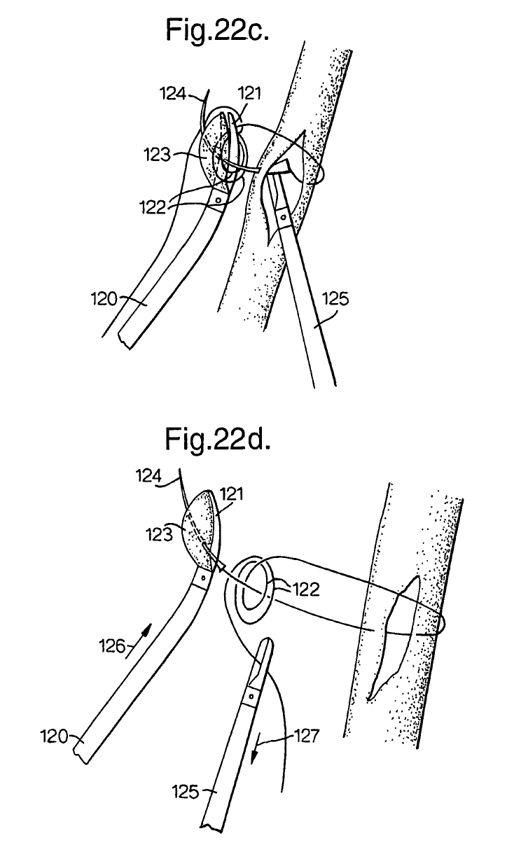

FIGS. 22a-22d are schematic views of successive steps in forming and tying at least one intracorporeal knot during suturing.

Although the present invention is exemplified in conjunction with techniques of laparoscopic suturing and ligation, the invention is not intended to be so limited. For example, it is intended that the invention will find application outside of uses relating to the human or animal body.

Referring to the Figures, wherein like numerals and symbols refer to like parts throughout, there is shown a laparoscopic needle delivery device 10. The laparoscopic needle delivery device 10 includes a generally elongage member in the form of a tubular rod 12 having a central lumen 14 for receiving a length of filamentary suture material 20, a first receiving means in the form of a needle carrying portion 16 for receiving and carrying a curved needle 30, and a second receiving means in the form of a loop carrying portion 18 for receiving and carrying at least one loop 22 of suture 20.

The tubular rod 12 has a tapered distal end 11, and may be formed from either a rigid plastics material or a biocompatible metallic material of approximately 5 mm diameter. In use, the rod 12 may be mounted adjacent to and in longitudinal alignment with a shaft of a grasping means, such as a set of straight or curved forceps or gripper, by a suitable attachment means such as windings of tape, a clip or similar mounting means. The tubular rod 12 can also be provided with a telescopic attachment by which the distal end 11 can be longitudinally translated and retracted when required.

In the case of a right handed surgeon, the laparoscopic needle delivery device 10 would be introduced concurrently with the shaft of the grasping means through a port disposed on a left hand side of a surgical site, whereas in the case of a left handed surgeon, the laparoscopic needle delivery device 10 would be introduced concurrently with the shaft of the grasping means through a port disposed on a right hand side of a surgical site.

A diameter of the central lumen 14 is chosen so as to readily receive the length of suture 20 as shown in FIG. 1. A distal end 24 of the suture 20 is endwise attached by conventional means to the needle 30, preferably to a curved needle 30.

The needle carrying portion 16 for receiving and carrying the needle 30 includes a pair of adjacent notches 17a, 17b that extend into the central lumen 14. In use, a free end 32 of the curved needle 30 is received in the notch 17a, then fed into the central lumen 14 until the free end 32 emerges from the adjacent notch 17b, as shown in FIG. 1. In this way, the needle 30 may be held in a desirable and stable arrangement on the laparoscopic needle delivery device 10, in general alignment with the longitudinal axis of the rod 12, when it is introduced through a port (not shown), which is a tubular device through which the illustrated equipment may be inserted into a body cavity. A second set of forceps or a grasping means, held and manipulated by the other hand of the surgeon, may then be used to unhook the needle 30 from the laparoscopic needle delivery device 10 and commence suturing, as shown in FIG. 2.

Alternatively, the needle carrying portion 16 may comprise a clip for receiving the curved needle 30 mounted on the tubular rod 12. Still further, the needle carrying portion 16 may comprise a tubular structure for receiving the needle 30 mounted longitudinally alongside the tubular rod 12.

Still yet further, the tubular rod 12 may be formed from a material, such as a plastics material, which can be pierced by the needle. The needle 30 may then be manipulated to pierce the tubular rod 12 so as to create a hole in the plastics material to hook itself to the tubular rod 12, and thus secure itself.

The loop carrying portion 18 for receiving and carrying at least one loop 22 of suture 20 includes a protruding member 19 extending substantially alongside the rod 12 such that a small gap is provided between the rod 12 and the protruding member 19 for receiving at least one loop 22 of suture 20. The protruding member 19 may take the form of a notch in the rod 12. Alternatively, the protruding member 19 may be formed to be retractable. In any case, the loop 22 of suture 20 may be held in a desirable and stable arrangement wholly to one side of the laparoscopic needle delivery device 10 when it is introduced through the port. A second set of forceps or a grasping means 33 held and manipulated by the other hand of the surgeon may then optionally be used to unhook the loop 22 from the laparoscopic needle delivery device 10. The forceps 33 are then used to unhook the needle 30 from the needle carrying portion 16 (see FIG. 2) and to pass the needle through the cut edges of a viscera or structure. After then releasing the needle 30, and passing the forceps 33 through the loop 22, the forceps may be used to re-grasp the needle and to pull it through the loop 22. If the loop 22 was not previously unhooked from the device 10 it can now be released from the loop carrying portion 18 or alternatively can be released by tensioning both ends of the suture thereby forming a double knot to secure the suturing, as shown in FIG. 3. In all cases, it will be apparent that the loop is released from the rod 12 whilst still formed as a loop.

As an alternative to having to release the needle 30 from the grip of the forceps 33 after passing the needle through the viscera or structure, the forceps may instead be passed through the loop 22 (whilst the loop is carried by the loop carrying portion 18) before being used to grip the needle 30 and remove it from the needle carrying portion 16. In this way the forceps 33 are already extending through the loop 22 when the needle is passed through the viscera or structure (not shown).

Preferably, a pair of loops 22 of suture is received in the loop carrying portion 18 to enable the formation of a double knot. Still more preferably, the pair of loops 22 is formed according to the following description as shown in FIGS. 4 to 8.

Referring to FIG. 4, two loops resembling a figure of eight shape are formed extracorporeally, wherein a first loop 220 is formed proximal to the distal end 24 of the suture 20 attached to the needle 30 and a second loop 230, extending in an opposing direction to the first loop 220, is formed proximal to the first loop 22. The distal end 24 of the suture 20 is arranged to be disposed anterior to a vertical limb 215 of the figure of eight shape, as is a proximal end 26 of the suture 20. The second loop 230 is then inverted so as to be adjacent and in parallel alignment with the first loop 220 so that the distal end 24, as well as the proximal end 26, of the suture 20 is disposed intermediate the first and second loops 220, 230, as shown in FIG. 5. This arrangement of the loops 220, 230 will be recognised as similar to that of a clove hitch knot, in which knot configuration the element to which the filamentary material is to be secured would normally be passed through the loops 220, 230 and the loops pulled tight. Instead, in the present situation it is envisaged that the pair of loops 220, 230 would be received in the loop carrying portion 18 of the laparoscopic needle delivery device 10 (in the manner shown in FIG. 1) by means of a grasping tool and introduced intracorporeally as described above. Once the distal end 24 has passed through the loops 220, 230, the loops 220, 230 can then be readily disengaged from the loop carrying portion 18 by a releasing mechanism or an appropriate grasping tool, or by pulling respective suture ends 24, 26. With this basic clove hitch structure of FIG. 5 it is important that the needle is passed through the loops from the back side (as drawn), i.e. passing first through the loop 220 and then through loop 230.

The loops 220 and 230 can be kept fixed temporarily by using a conventional biocompatible biodegradable glue. This is particularly suitable for sutures formed from synthetic suture materials, such as polyglycolic acid. The loops 220 and 230 may be preformed before insertion into the port, wherein the loops 220 and 230 are “weakly glued” together by the desired adhesive.

It will be understood that the pair of loops 220, 230 described above may also be introduced intracorporeally through the port with a conventional grasping tool.

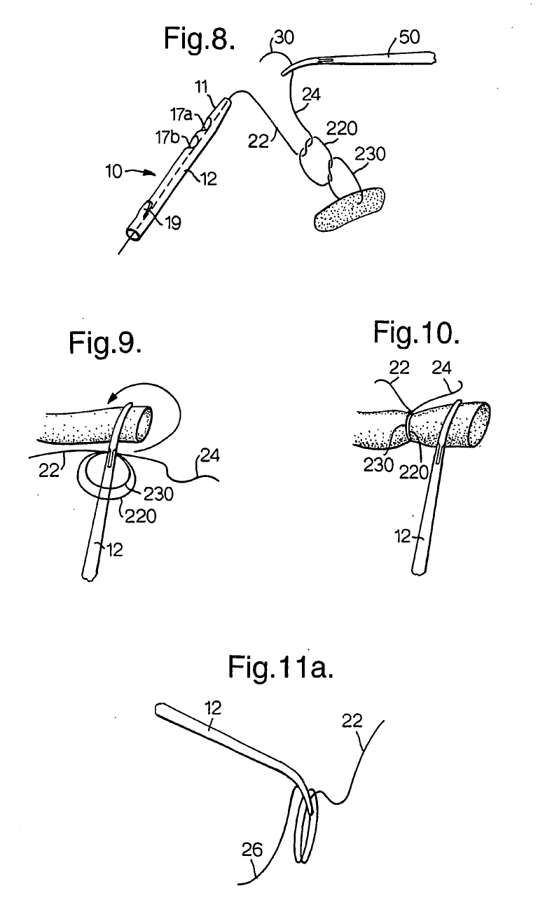

The needle 30 carrying the distal end 24 of the suture, after passing through cut edges of the viscera, may be pulled through the pair of loops 220, 230 as shown in FIG. 7. The distal and proximal ends 24, 26 of the suture 20 can thus be pulled simultaneously by grasping tools, thereby allowing loops 220, 230 to form a double knot to secure the suturing, as shown in FIG. 8. Further securing with additional knots can then be formed in the conventional fashion.

As an alternative to suturing, the pair of loops 220, 230 may be threaded over a terminal end of a cut tubular structure, such as a blood vessel, such that the terminal end of the tubular structure extends through the loops 220, 230 as shown in FIG. 9. The distal and proximal ends 24, 26 of the filamentary material 20 can then be pulled simultaneously by grasping tools, thereby tightening loops 220, 230 and affording ligation of the terminal end of the tubular structure, as shown in FIG. 10. In this way, the clove hitch structure of the loops 220, 230 (discussed above in conjunction with FIG. 5) is employed as a conventional clove-hitch knot. Further securing with additional knots can then be formed in the conventional fashion.

Ligation of a continual tubular structure, such as a blood vessel, may also be achieved by positioning the pair of loops 220, 230 (in the basic clove hitch structure of FIG. 5) adjacent the tubular structure to be ligated, and passing an end of the filamentary material 20 around the tubular structure to be ligated, and then passing that end through the pair of loops 220, 230. If it is the proximal end 26 of the filamentary material 20 that is passed around the tubular structure, then that proximal end must return through the loop 230 first and then through the loop 220. If, though, it is the distal end 24 which is passed around the tubular structure, then that distal end must return through the loop in the other direction, i.e. pass first through the loop 220 and then through the loop 230. The distal and proximal ends 24, 26 of the suture 20 can then be pulled simultaneously by grasping tools, thereby tightening loops 220, 230. Further securing with additional knots can then be formed in the conventional fashion.

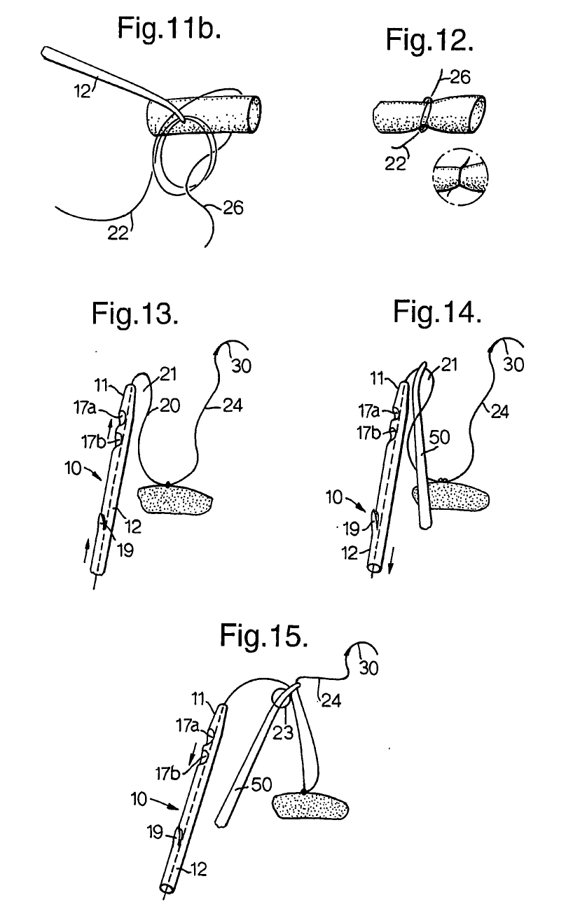

Instead of using a knot based on a clove hitch to ligate a tubular structure, one may be used based on a so-called surgeon's knot. FIG. 11a illustrates the general arrangement of the loops 220, 230 of the filamentary material prior to the loops being offered up to the tubular structure to be ligated. In essence, the loop structure may comprise two simple loops arranged in the form of a helical coil. When these loops 220, 230 are presented by the surgeon to the posterior side of the structure to be ligated, the proximal end 26 of the filamentary material (which is closest to the surgeon) is passed around the structure to be ligated and then passed through the anterior face of the loops 220, 230. By then tensioning the loops the ligature can be formed and knotted, as shown in FIG. 12. Alternatively, the anterior end of the material may be passed around the structure and be passed through the loops from the posterior side.

The inventor of the present invention has found that it is advantageous to provide the one end of the filamentary material 20 with a small weight. For example, by providing the distal end 24 with a weight, when the distal end 24 is passed over the tubular structure, the weight falls under the influence of gravity to a relatively lower position, making it easier to grasp. It is found that the distal end 24 of the suture 20 is thus conveniently disposed underneath the blood vessel or tube and may then be easily grasped and pulled through the pair of loops 220, 230, without any requirement for further manipulation, thus saving time during the suturing process.

It is envisaged that the weight may be tear-drop shaped and formed from a metallic material, or other dense rigid material that is readily sterilised, or alternatively, a biodegradable material.

The laparoscopic needle delivery device 10 of the present invention can be advantageously and readily used in the intracorporeal formation of further loops/knots for securing the suture knot formed on the viscera or tubular structure. Referring to FIGS. 13 to 15, in which an existing suture knot is shown, the laparoscopic needle delivery device 10 can be forwardly translated in relation to the viscera to form a half loop 21 in the provimal portion of the filamentary suture material 20 between the device 10 and the existing knot. A curved needle holder 50, for example in the form of a grasping tool, such as a pair of forceps, is placed inside the half loop from the right hand side of the loop 21, as shown in FIG. 14. The needle delivery device 10 is retracted to form a complete loop and the needle bolder 50 is caused to grasp the distal portion 24 of the suture 20. The needle holder 50 is then withdrawn through the loop 23 so formed, as shown in FIG. 15, so pulling the distal end of the suture 20 back through the loop 23 to form another knot on top of the previous suture knot. The knot so formed is executed by the surgeon with merely a simple backwards and forwards motion of the laparoscopic needle delivery device 10 and the grasping tool held in the opposing hand. No rotational movement, which is a difficult technique to perform intracorporeally, is required. The formation of the half loop and complete loop may, however, be performed by rotation of the device 10 about its longitudinal axis, instead of by longitudinal movement.

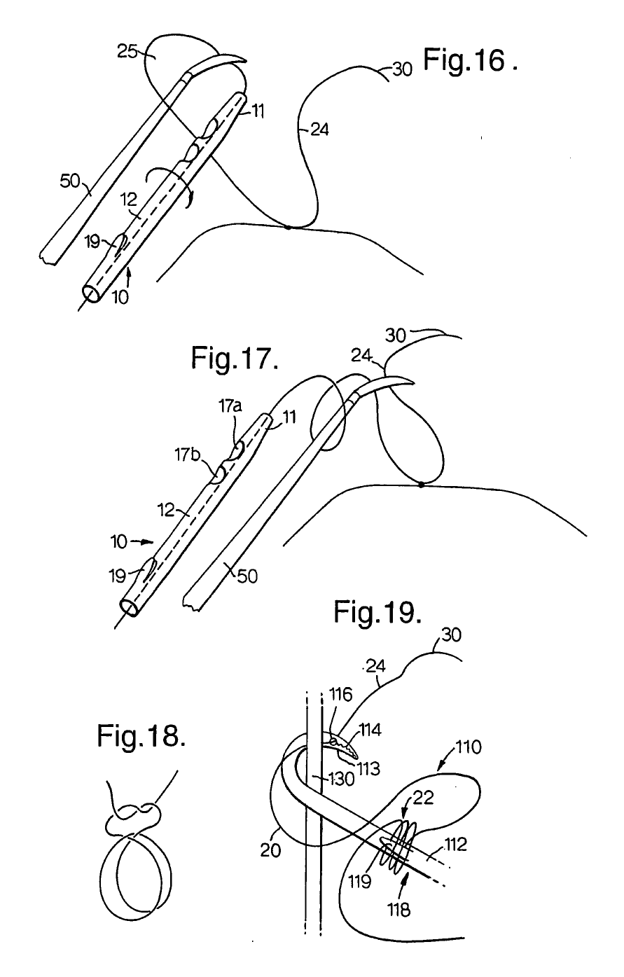

An intracorporeal loop 22 and subsequent knot may also be conveniently made and tied by rotating the laparoscopic needle delivery device 10 or grasping forceps to hold the reverse loop 25. Referring to FIGS. 16 and 17, a reverse loop 25 can be formed by disposing the tubular rod 12 of the device 10 anterior to the suture 20, then rotating the tubular rod 12 in a clockwise direction about the rod's own longitudinal axis whilst simultaneously forwardly translating the tubular rod 12. The grasping tool 50 is passed from the left hand side of the tubular rod 12 into the reverse loop 25 so formed and caused to grasp the distal end 24 of the suture 20. The grasping tool 50 is then withdrawn through the reverse loop 25, so pulling the distal end 24 of the suture 20 back through the reverse loop 25 to form a reverse knot, as shown in FIG. 17. In this way, two additional knots over an original double knot should make the knot very secure.

It is envisaged that the loops of a “surgeon's knot” may also be formed extracorporeally, whereupon the loops may be received in the loop carrying portion 18 of the laparoscopic needle delivery device 10, delivered through the port to the area of interest and secured according to conventional means. In addition, a simple knot for use as an additional knot, on top of one of the above described knots, may be formed extracorporeally, as shown in FIG. 18.

Referring to FIG. 19 there is shown a laparoscopic suturing device 110 for ligating blood vessels or tubular structures. The laparoscopic suturing device 110 includes an elongate member 112 having a hooked distal end 114 for receiving a length of blood vessel 130, an aperture 116 disposed in the hooked distal end 114 for receiving a length of suture 20, and a receiving means 118 for receiving at least one loop of suture 22. The device 110 also includes a hinged grasping means 113 comprised of two hinged jaws that can be opened or shut by pressing or releasing an operative extracorporeal handle.

The elongate member 112 is formed from either a rigid plastics material or a biocompatible metallic material of approximately 5 mm diameter. In the case of a right handed surgeon, the laparoscopic suturing device 110 would be introduced through a port disposed on a left hand side of a surgical site, whereas in the case of a left handed surgeon, the laparoscopic suturing device 110 would be introduced through a port disposed on a right hand side of a surgical site.

A diameter of the aperture 116 is chosen so as to readily receive the length of suture 20 as shown in FIG. 19. It is envisaged that the aperture 116 is formed from opposing semicircular recesses disposed in each jaw of the grasping means 113, wherein the recesses are disposed adjacent the hinge of the jaws.

The receiving means 118 for receiving at least one loop 22 of suture 20 includes a protruding member 119 extending substantially alongside the elongate member 112 such that a small gap is provided between the elongate member 112 and the protruding member 119 for receiving at least one loop 22 of suture 20. The protruding member 119 may take the form of a notch in the rod 12. Alternatively, the protruding member 119 may be formed to be retractable. In any case, the loop 22 of suture 20 may be held in a desirable and stable arrangement on the laparoscopic suturing device 110 when it is introduced through the port. A second set of forceps or a grasping ans held and manipulated by the alternative hand of the surgeon may then be used to unhook the loop 22 from the laparoscopic suturing device 10.

In use, a length of blood vessel 130 is received in the hooked distal end 114 whereupon the suture end 24 may be pulled through the loop 22 which will be detached from the receiving means 118 of the laparoscopic suturing device 110, thereby forming a knot to ligate the blood vessel 130, as shown in FIG. 19. Additional knots can be tied as described with reference to FIGS. 13, 14, 15, 16 and 17. After the suture 20 has been cut, the hooked distal end 114 may disengage with the length of blood vessel 130. In this way, ligation of blood vessels 130 may be conveniently performed in a very simple action.

The present invention also relates to the ligation or tying off of tubular structures using conventional instruments.

FIGS. 20a-20d illustrate a sequence of events in which a tubular structure is ligated using as few as one gripping tool such as a set of forceps 100, although more than one such tool may be used.

Although in the FIGS. 20a-20d sequence a pair of loops 101 is shown as being provided, the technique will work with a single loop. In the illustrated arrangement the loops 101 have the basic surgeon's knot type structure illustrated in FIG. 11a and the distal end of the filamentary material 102 is shown as being provided with a teardrop weight 103 of the sort discussed above. As shown in FIG. 20b, this weight 103 is draped over the tubular structure 104 to be ligated and the weight 103 is allowed to dangle.

If the technique is being performed with a single grasping tool 100, the loops 101 then need to be positioned underneath the weight 103 so as to enable the weight 103 to pass through both loops 101. The technique is, however, easier to perform if a second gripping tool (not shown) is, in the FIG. 20b condition, extended through the loops to grasp the distal portion of the filamentary material 102 behind the weight 103. In this way the distal end of the filamentary material 102 can more readily be drawn through the loops 101, as shown in FIG. 20c.

Once the distal end of the filamentary material 102 has been passed through the loops 101, the gripping tool 100 can be released from the loops 101 and the loops tightened so as to ligate the vessel and to form a knot. It will be appreciated that this tightening of the loops can most readily be achieved in the preferred situation discussed above, in which two gripping tools are employed.

It will be appreciated that basic knot configurations other than the surgeon's knot may be employed.

The sequence of events illustrated in FIGS. 21a-21e illustrates an alternative arrangement employing the basic surgeon's knot type structure illustrated in FIG. 11a. The instrument 110 can be introduced into a body cavity through an appropriate port. The instrument 110 includes comprises a generally elongate member 111, provided at its curved distal end with a gripper 112 comprising a pair of hinged jaws which can operated from outside of the body. The generally elongate member 111 is telescopically slidable within a sleeve 113. Provided adjacent to the sleeve 113 is a tubular structure 114 carrying the proximal portion of a length of filamentary material 115.

The distal portion of the filamentary material 115 has at least one loop 116 formed therein. In the illustrated arrangement a pair of loops is provided. These loops may be arranged in the clove hitch configuration illustrated in FIG. 5 or, as shown, in the basic surgeon's knot configuration illustrated in FIG. 11b, or indeed in any other suitable basic knot configuration. In the case of the surgeon's knot configuration, the most distal portion of the filamentary material 115 extends from the more proximal of the two loops 116, in other words from the posterior side which is the side closest to the surgeon. As can be seen in FIG. 21a, the gripper 112 grips the filamentary material 115 between the loops 116 and the distal tip of the filamentary material which is provided with a weight 117.

By manipulating the instrument 110 the distal tip of the filamentary material 115 and the weight 117 may be passed over the tubular structure 118 to be ligated, whilst being held in the gripper 112, as shown in FIG. 21a. The gripper 112 is then released and re-positioned underneath the tubular structure 118, at which position it is used to re-grip the distal end of the filamentary material 115, as shown in FIG. 21b. By then withdrawing the generally elongate member 110 in the proximal direction relative to the sleeve 113 the sleeve causes the loops 116 to travel in the distal direction down the generally elongate member 111 and down the gripper 112, causing the distal tip of the filamentary material 115 to pass through the loops 116 from the anterior side, as shown in FIG. 21c. By then pushing the generally elongate member 111 in one direction (whilst holding on to the distal top of the filamentary material 115 with the gripper 112), and pulling and withdrawing the tubular structure 114 in the other direction (as shown in FIG. 21d), the loops 116 can be tensioned, thereby causing the structure 118 to be ligated and knotting the filamentary material.

By then advancing the instrument 110, to provide some slack in the filamentary material 115 between the tubular structure 114 and the knot, and then rotating the tubular structure 114, a further loop 119 can be formed in the filamentary material, enabling the grippers 112 to be again used to manipulate the distal end of the filamentary material 115 and the weight 117 through the loop 119 so as to enable the formation of a further knot as shown in FIG. 21e. Alternatively, the further loop 119 can be formed by using the grippers 112 to grip the filamentary material 115 proximally of the existing knot (for example 10 cm proximally of the knot) and then rotating the instrument 110 so as to form the loop 119

In this way a single instrument 110 can be used to tie knots intra-corporeally.

In the arrangements illustrated in FIGS. 20 and 21 the proximal end of the filamentary material 115 remains free and outside the port and body cavity. As in the earlier embodiments it is intended that the initial loops 101, 116 will be pre-formed outside the body cavity, spaced back from any distal weight 117, for example by approximately 5 to 7 cms. The loops of filamentary material can then be introduced into the body cavity through an appropriate port.

It will be appreciated that single or double knots can be achieved according to the type and number of loops 101, 116 employed.

FIGS. 22a-22d illustrate a sequence of events in the use of a needle device suitable for use in laparoscopic surgery. The device comprises a generally elongate member 120, made up of a straight stem and a curved distal end. The distal end comprises a gripper 121 for gripping loops of filamentary material, the gripper comprising a pair of hinged jaws. As will become clear below, the jaws of the gripper 121 can be used to releasably carry the loops 122 of filamentary material.

Provided on the back of one of the jaws of the gripper 121 is a needle carrying portion in the form of a cushion 123 of resilient sponge-like material. The cushion material 123 can be a small piece of sponge material wrapped in a suitable cloth, such as prolene mesh or similar. The cushion material can be pierced by the needle 124 so as to retain the needle relative to the gripper 121 in a plurality of different orientations. FIG. 22a shows the needle 124 being carried in a longitudinal orientation, in which the needle is generally in the same plane as the longitudinal axis of the elongate member. In FIGS. 22c and 22d the needle 124 is shown as being accommodated by the cushion 123 in a transverse orientation, in which the needle 124 is generally perpendicular to that longitudinal axis.

In use, at least one loop 122, ideally a plurality of loops as shown, is/are formed extra corporeally and gripped by the gripper 121, as shown in FIG. 22a. As with the earlier embodiments, the loops 122 may be formed in the basic clove hitch configuration or the surgeon's knot configuration, or indeed any other suitable basic knot configuration. The needle 124, attached to the distal end of the filamentary material, is inserted into the cushion material 123 extracorporeally to assume the longitudinal orientation shown.

The instrument, in the condition shown in FIG. 22a, can then be pushed through an appropriate sized port during laparoscopic surgery. Once inside the body cavity the instrument can be rotated to the orientation shown in FIG. 22b and the needle 124 can easily be grasped by a gripping tool, such as a pair of forceps 125, the distal end of which is illustrated in FIG. 22b. After pulling the needle 124 from the cushion 123 the forceps 125 can then be used to guide the curved needle 124 through the cut tissues, as shown in FIG. 22c. During this movement the loops 122 continue to be held by the gripper 121.

The distal tip of the curved needle 124, after its passage through the cut tissues, is guided back through the loops 122 and embedded in the cushion 123, as shown in FIG. 22c. Contrary to its orientation in FIG. 22a, in FIG. 22c the needle 124 is oriented transversely, i.e. with its axis or plane generally transverse to the axis or plane of the elongate member 120. The loops 122 of filamentary material may then be released by the gripper 121 to pass down the needle 124. Once the needle 124 is embedded in the cushion 123 the forceps 125 can be released from the needle 124 and used instead to grip the filamentary material proximally of the loops 122, as shown in FIG. 22d. By then moving the instrument 120 away from the stitched tissue, for example in the direction of arrow 126 in FIG. 22D, and simultaneously moving the forceps 125 in the opposite direction, for example in the direction of arrow 127 in FIG. 22d, the loops 122 may be tensioned to form the desired knot.

The transverse orientation of the needle relative to the instrument 120 is highly advantageous. As surgeons will know, trying to get a needle in this transverse position in a body cavity is very difficult. Once in this transverse orientation, and after formation of the initial suture, the needle 124 may be used to carry on continuous suturing and further knotting.

It is envisaged that the instruments might be made of biocompatible synthetic non-reusable material or of reusable stainless steel.

1. A laparoscopic needle delivery device, comprising a generally elongate member, the member having a needle carrying portion for releasably carrying a needle and a loop carrying portion for releasably carrying at least one loop of filamentary material, said loop carrying portion being constructed and arranged to enable said at least one loop to be released from the elongate member whilst still formed as a loop.

2. A device as claimed in claim 1, wherein the filamentary material is suture material and has a more proximal portion and a more distal portion, said elongate member has a hollow central lumen to receive said more proximal portion of said suture material, and said at least one loop is formed in said more distal portion of said suture material.

3. A device as claimed in claim 2, wherein the more distal portion of said suture material extends out of the central lumen at the distal tip of the elongate member.

4. A device as claimed in claim 1, wherein the needle carrying portion of the elongate member has a longitudinal axis and the needle carrying portion is constructed and arranged to allow the needle to be carried in an orientation in which the needle is generally aligned with that longitudinal axis.

5. A device as claimed in claim 1, wherein the needle carrying portion comprises one or more pre-formed apertures provided in a wall of the elongate member.

6. A device as claimed in claim 1, wherein the needle carrying portion of the elongate member includes material which can be penetrated by a needle, enabling the needle to be inserted into said material so as to create a hole in said material thereby to be carried by the elongate member.

7. A device as claimed in claim 1, wherein the loop carrying portion is provided to one side of the elongate member so that said at least one loop is wholly positioned to that side of the elongate member.

8. A device as claimed in claim 1, wherein the loop carrying portion is arranged to carry said at least one loop alongside the elongate member.

9. A device as claimed in claim 1, wherein the elongate member has a longitudinal axis and the loop carrying portion is arranged to carry said at least one loop adjacent to said axis.

10. A device as claimed in claim 9, wherein said carried at least one loop is not coaxial to said longitudinal axis.

11. A device as claimed in claim 1, wherein said loop carrying portion comprises a projection under which said at least one loop can be carried to be carried by said elongate member.

12. A combination of: a device as claimed in claim 1; said needle; and a length of said filamentary material.

13. A combination as claimed in claim 12, wherein the needle is attached to the distal end of the length of filamentary material, the length of filamentary material has said at least one loop formed therein at a position spaced from said distal end, said at least one loop is engaged with said loop carrying portion so as to be carried thereby and said needle is engaged with said needle carrying portion so as to be carried thereby.

14. A method of using the combination of claim 13 to form a suture, the method comprising releasing the needle from the needle delivery device, passing the needle through the elements to be sutured, passing the needle through the centre of said at least one loop and tensioning said at least one loop to cause said loop to contract to form a knot in the filamentary material.

15. A method as claimed in claim 14, wherein said at least one loop is removed from the loop carrying portion before the needle is passed through the loop's centre.

16. A method as claimed in claim 14, wherein said at least one loop is being carried by the loop carrying portion when the needle is passed through the loop's centre.

17. A method as claimed in claim 1, wherein the elements to be sutured are intracorporeal.

18. A laparoscopic device for use in ligating a tubular structure, the device comprising an elongate member having a generally hook-shaped distal portion to enable the tubular structure that is to be ligated to be received in said portion, said elongate member further comprising a loop carrying portion for releasably carrying at least one loop of filamentary material, said loop carrying portion being constructed and arranged to enable said at least one loop of material to be released from the elongate member whilst still formed as a loop.

19. A device as claimed in claim 18, wherein the loop carrying portion is provided to one side of the elongate member so that said at least one loop is wholly positioned to that side of the elongate member.

20. A device as claimed in claim 18, wherein the loop carrying portion is arranged to carry said at least one loop alongside the elongate member.

21. A device as claimed in claim 18, wherein the elongate member has a longitudinal axis and the loop carrying portion is arranged to carry said at least one loop adjacent to said axis.

22. A device as claimed in claim 21, wherein said carried at least one loop is not coaxial to said longitudinal axis.

23. A device as claimed in claim 18, wherein said loop carrying portion comprises a projection under which said at least one loop can be wedged to be carried by said elongate member.

24. A combination of: the device of claim 18, and a length of said filamentary material.

25. A combination as claimed in claim 24, wherein said length of filamentary material has said at least one loop formed therein and said at least one loop is engaged with said loop carrying portion so as to be carried thereby.

26. A combination as claimed in claim 24, wherein the length of filamentary material is provided with a weight in the region of its distal end and said at least one loop is spaced proximally from said weight.

27. A method of using the combination according to claim 24 to ligate a tubular structure, the method comprising engaging with said generally hook- shaped distal portion the tubular structure to be ligated, passing the distal end of the filamentary material around the tubular structure and back through the centre of said at least one loop and tensioning said at least one loop to ligate the tubular structure and to form a knot.

28. A method as claimed in claim 27, wherein the tubular structure is intracorporeal.

29. A device or method as claimed in claim 1, wherein said at least one loop of filamentary material comprises part of a plurality of adjacent loops-formed in said filamentary material.

30. A device as claimed in claim 29, wherein said plurality of adjacent loops form the basic structure of a clove hitch knot.

31. A device as claimed in claim 29, wherein said plurality of adjacent loops form the basic structure of a surgeon's knot.

32. A device as claimed in claim 1, wherein said plurality of loops are constructed and arranged so that, when the loops are tensioned, they will contract to form at least one knot in the filamentary material.

33. A device as claimed in claim 32, wherein said plurality of loops are constructed and arranged so that, when the loops are tensioned, they will contract to form a double knot.

34. A laparoscopic needle delivery device, comprising a generally elongate member, the member being provided with a loop carrying portion for releasably carrying at least one loop of filamentary material and a needle carrying portion for releasably carrying a needle in both a longitudinal orientation, in which the needle is generally aligned with the longitudinal axis of the elongate member, and a transverse orientation, in which the needle is generally perpendicular to said longitudinal axis.

35. A device as claimed in claim 34, wherein the loop carrying portion comprises a gripper for gripping the loops.

36. A device as claimed in claim 35, wherein the gripper comprises at least one hinged jaw.

37. A device as claimed in claim 36, wherein the loop carrying portion is provided on said at least one hinged jaw.

38. A device as claimed in claim 34, wherein the needle carrying portion of the elongate member includes material which can be penetrated by a needle, enabling the needle to be inserted into said material so as to create a hole in said material thereby to be carried by the elongate member.

39. A device as claimed in claim 38, wherein said material comprises a resilient sponge-like material.

40. A device as claimed in claim 34, wherein the generally elongate member has a curved distal end on which at least one of said loop carrying portion and needle carrying portion is provided.

41. A combination of: a device as claimed in claim 34, said needle; and a length of said filamentary material.

42. A combination as claimed in claim 41, wherein the needle is attached to the distal end of the length of filamentary material, the length of filamentary material has said at least one loop formed therein at a position spaced from said distal end, said at least one loop is engaged with said loop carrying portion so as to be carried thereby and said needle is engaged with said needle carrying portion so as to be carried thereby.

43. A method of using the combination of claim 41 to form a suture, the method comprising: removing said needle from an initial needle position in said needle carrying portion; passing the needle through the structure to be sutured, the centre of said at least one loop, and back into the needle carrying portion to assume a subsequent needle position; releasing said at least one loop from said loop carrying portion to pass down the needle; and tensioning said at least one loop to form and knot the suture.

44. A method as claimed in claim 43, wherein in said initial needle position said needle is in said longitudinal orientation, and in said subsequent needle position said needle is in said transverse orientation.

45. A method as claimed in claim 41, wherein said needle is manipulated relative to said laparoscopic needle delivery device using a gripping tool.

46. A method as claimed in claim 43, wherein the tubular structure is intracorporeal.

47. A device as claimed in claim 34, wherein said at least one loop of filamentary material comprises part of a plurality of adjacent loops formed in said filamentary material.

48. A device as claimed in claim 47, wherein said plurality of adjacent loops form the structure of a clove hitch knot.

49. A device as claimed in claim 47, wherein said plurality of adjacent loops form the basic structure of a surgeon's knot.

50. A device as claimed in claim 42, wherein said plurality of loops are constructed and arranged so that, when the loops are tensioned, they will contract to form at least one knot in the filamentary material.

51. A device as claimed in claim 50, wherein said plurality of loops are constructed and arranged so that, when the loops are tensioned, they will contract to form a double knot.

52. A method of ligating a tubular structure, the method comprising: providing a length, of filamentary material having at least one loop formed therein spaced proximally from the filamentary material's distal end; positioning said at least one loop adjacent the tubular structure to be ligated; passing the distal end of the filamentary material around the tubular structure and then through the centre of said at least one loop; and tensioning said at least one loop to ligate the tubular structure and to-form a knot in the filamentary material.

53. A method as claimed in claim 52, wherein said at least one loop is continuously gripped by a first gripping tool up to and including the step of passing the distal end of the filamentary material through the centre of said at least one loop and is released from said first gripping tool prior to said loop tensioning step.

54. A method as claimed in claim 53, wherein the first gripping tool, after release at said at least one loop, is used to grasp the filamentary material to one side of said at least one loop to enable said at least one loop to be tensioned during said loop tensioning step.

55. A method as claimed in claim 53, wherein the distal end of the filamentary material is passed through the centre of said at least one loop by the action of inserting a second gripping tool through said at least one loop, grasping the distal end of the filamentary material and pulling the distal end of the filamentary material back through said at least one loop.

56. A method as claimed in claim 52, wherein said at least one loop is formed coaxially around an instrument having a gripper at its distal end.

57. A method as claimed in claim 56, wherein the gripper is used to grip the filamentary material between said at least one loop and the filamentary material's distal end so as to enable the distal end of the filamentary material to be positioned on the opposite side of the tubular structure to said at least one loop.

58. A method as claimed in claim 57, wherein the gripper is used to position the distal end of the filamentary material on said opposite side of the tubular structure, the gripper is released from the filamentary material, repositioned on the other side of the structure, and then used to re-grip the distal end region of the filamentary material to guide the distal end of the filamentary material back through the centre of said at least one loop so as to tension said at least one loop.

59. A method as claimed in claim 56, wherein said at least one loop is caused to move distally along said instrument towards said gripper after said gripper has been used to rejoin the filamentary material.

60. A method as claimed in claim 56, wherein a proximal end of said length of filamentary material is associated with a sleeve around said gripper, whereby said at least one loop is tensioned by longitudinal relative displacement of said sleeve and said gripper.

61. A method as claimed in claim 52 wherein the distal end of the filamentary material is provided with a weight.

62. A method of forming an additional knot to secure a prior knot formed in a continuous length of filamentary material, a proximal portion of the filamentary material extending between a generally elongate first instrument and the prior knot and a distal portion of said filamentary material extending between the prior knot and the distal end of the filamentary material, the method comprising: a) manipulating the first instrument and a second generally elongate instrument to form a loop in the proximal portion of the filamentary material, with said second generally elongate instrument extending through said loop; b) gripping the distal portion of the filamentary material using said second instrument; c) withdrawing the second instrument through said loop to draw the distal portion of the filamentary material through said loop; and d) tensioning said loop to form said additional knot on top of said prior knot.

63. A method as claimed in claim 62, wherein the step a) involves the following sub-steps: e) manipulating the first instrument to form said loop in said proximal portion of the filamentary material; and f) extending the second instrument through said loop.

64. A method as claimed in claim 63, wherein formation of said loop is achieved by rotating said first instrument.

65. A method as claimed in claim 63, wherein formation of said loop is formed by a relative longitudinal forward and backward movement of the first and second instruments.

66. A method as claimed in claim 52 wherein said method is performed intracorporeally.

67. A method as claimed in claim 52 wherein said method is performed on a structure not involving the human or animal body.
